(19) United States
(12) Patent Application Publication
(10) Pub. No.: US 2002/0127282 A1
(43) Pub. Date: Sep. 12, 2002

(54) Multivalent silver pharmaceuticals

(75) Inventor: Marvin S. Antelman, Rehovot (IL)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: N. Jonas & Co., Inc. a/k/a N. Jonas & Co., Bensalem, PA

(21) Appl. No.: 10/072,520

(22) Filed: Feb. 5, 2002

Related U.S. Application Data
(63) Continuation of application No. 07/802,478, filed on Dec. 5, 1991.

(51) Int. Cl. A61K 33/38

(52) U.S. Cl. 424/618

(57) ABSTRACT

Novel pharmaceuticals are described based on multivalent silver compounds containing Ag(II) or Ag(III) capable of killing pathogenic gram positive and negative bacteria, fungi and algae such as E. coli, Staphylococcus aureus and epidermidis, and Candida albicans. The efficacy of these compounds is enhanced by oxidizing agents such as persulfates. They can also be utilized to preserve pharmaceutical, cosmetic and chemical specialty products against these pathogens.

### CROSS REFERENCE TO RELATED APPLICATION

This patent application is a continuation of copending U.S. patent application Ser. No. 07/802,478, filed on Dec. 5, 1991, entitled “Multivalent Silver Pharmaceuticals.” The entire disclosure of U.S. patent Ser. No. 07/805,478 as filed is incorporated herein by reference.

### BACKGROUND OF THE INVENTION

The present invention relates to the employment of multivalent silver compounds and their utilization as pharmaceuticals. This invention, however, relates more particularly to the utilization of bactericidal, viricidal, algicidal and fungicidal activity exhibited by this class of compounds in pharmaceuticals. The compounds involved range from divalent to trivalent silver compositions including mixed crystals in which silver is present in a multivalent state along with monovalent silver, such as tetrasilver tetroxide which contains two monovalent silver ions and two trivalent ions per molecule. The utilization of water-soluble divalent silver (Ag II) complex bactericides is the subject of U.S. Pat. No. 5,017,295 of the present inventor.

I have also been granted U.S. Pat. Nos. 5,073,382, 5,078,902, 5,089,275, and 5,098,582 which all deal with Ag(II) bactericides but more particularly with (respectively), alkaline pH, halides, stabilized complexes, and the divalent oxide.

The main thrust of all of these patents, with the exception of the alkaline pH patent (U.S. Pat. No. 5,078,382) which is entitled DIVALENT SILVER ALKALINE BACTERICIDE COMPOSITIONS, is the utilization of these multivalent silver compounds as sanitizers in bodies of water ranging from swimming pools to industrial cooling towers. The latter patent, however, teaches compositions capable of being used as bactericides in such applications as food and dairy cleaners and surgical scrubbing soaps. Whereas said multivalent silver compounds were utilizable in the aforementioned applications, they were not evaluated under more stringent conditions demanded for their utilization as bactericides in pharmaceuticals. Furthermore, their efficacy as fungicides and algicides was not evaluated. Accordingly, a comprehensive testing program was begun involving said compounds which involved not only testing for efficacy but evaluating the compounds' systemic and acute dermal toxicity in animals as well as their eye and primary dermal irritation. Select compositions of said compounds proved to be nontoxic, nonirritating, and effective against all the aforementioned pathogenic classes.

### OBJECTS OF THE INVENTION

The main object of this invention is to provide for multivalent silver compound compositions suitable for utilization in pharmaceuticals which are capable of killing bacteria, fungi, viruses and algae, said capability requiring the compositions to achieve 100% kills of specific pathogens which are growing in a nutrient indigenous to the specific pathogen in question.

Another object of the invention is to provide for said multivalent silver compositions capable of the aforementioned antipathogenic pharmaceutical functions for veterinary or human application without having toxic side effects, or causing eye or dermal irritations.

Still another object of the invention is to utilize said multivalent silver compositions to preserve pharmaceutical, cosmetic and chemical specialty products against said pathogens.

Other objects, features and advantages of the invention shall become apparent as the description thereof proceeds when considered in connection with the accompanying specific examples.

### SUMMARY OF THE INVENTION

This invention relates to multivalent silver compound compositions capable of destroying gram positive and gram negative bacteria as well as fungi, viruses and algae in such a manner as to meet the stringent protocol requirements indigenous to pharmaceutical products. It also relates to the utilization of said compositions as preservatives against the aforementioned pathogens in cosmetic, pharmaceutical and chemical specialty products.

The multivalent silver compound compositions which are the subject of this invention are based on specific divalent and trivalent silver compounds including compounds containing silver in both a monovalent and multivalent state which have been described in the inventor's aforementioned patents. The divalent silver compounds are enumerated as follows:

1. Phosphate complexes.

2. Fluoborate complexes.

3. Chloride.

4. Bromide.

5. Iodide.

The special mixtures are as follows:

1. Divalent silver phosphate calcium sulfate adducts.

2. Divalent silver nitrate calcium sulfate adducts.

3. Divalent silver phosphate borax adducts.

Also included is what was referred to in my U.S. Pat. No. 5,098,582 as divalent silver oxide, which is the popular name of the compound.

The Merck Index (11th Edition) lists this compound (number 8469) as Ag(II) Oxide, and lists its formula as Ago. It then further states that “It is actually a silver (I)-(III) oxide.” The consensus of current chemical literature is that the actual formula is Ag₄O₄. A further elucidation of this compound is in order as it has been found superior to all the other compounds and thus is the subject matter of the preferred embodiments of this invention. The compound is prepared via the reaction of silver nitrate with sodium or potassium peroxydisulfate according to the following equation:

4AgNO₃+2Na₂S₂O₃+8NaOH═Ag₄O₄+4Na₂SO₄+4NaNO₃+4H₂O

As for the more important literature references relating, to the tetroxide formula for this compound, there are J. A. NcMillan's studies appearing in Inorganic Chemistry 13, 28 (1960); Nature, Vol. 195, No. 4841 (1962); and Chemical Reviews 1962, 62, 65. Furthermore, A. J. Salkind's studies involving neutron diffraction with his coworkers (J. Ricerca Sci. 30, 1034 1960) probed the Ag(III)/Ag(I) nature of this molecule and states in his classic entitled Alkaline Storage Batteries (Wiley 1969) coauthored with S. Uno Falk that the formula is depicted by Ag₄O₄ (P. 156). That same year a scientific communication appeared in Inorganic Nuclear Chemistry Letters (5, 337) authored by J. Servian and H. Buenafama which maintained that their neutron diffraction studies also confirmed the tetroxide lattice and the presence of Ag(III) and Ag(I) bonds in the lattice, a conclusion also reported previously by Naray-Szahn and Argay as a result of their x-ray diffraction studies (Acta Cryst. 1965,19, 180).

Said aforementioned multivalent silver moieties were initially screened for gram positive and negative efficacy. They were all effective at concentrations of about 1 PPM at giving 100% kills of these bacteria in standard tests designed for evaluating such efficacy under stringent conditions. Select compounds of said moieties were also evaluated against algae utilizing Chorella species incubated for 10 days in algae nutrient. The divalent silver phosphate and fluoborate complexes and multivalent silver tetroxide were effective at about 1 PPM in effecting 100% kills of algae within 5 minutes. The performance of these compounds was enhanced in the presence of various oxidizing agents, especially the persulfates. Indeed, in most cases there was no efficacy without the presence of a persulfate. Now the entire gamut of said silver moieties were systematically examined for the purpose of finding the most suitable compounds for further study. The divalent silver acid complexes were eliminated because they were found to stain the skin at low concentrations. A yellow insoluble phosphate precipitated from the acid divalent complex by elevating the pH was found to be nonstaining to skin and pathogenically active. However, after six months, the compound was found to have lost its efficacy. The divalent halides, on the other hand, involved too many manufacturing steps when compared to the efficacy of the silver tetroxide. The same was true for the divalent silver calcium sulfate and borax adducts. Accordingly the bulk of the studies and evaluations which constitute the subject matter of this invention involve silver tetroxide. It was evaluated at 0.5 and 1.0 PPM on gram positive and negative cultures in the presence of 10 PPM sodium persulfate. It killed 100% of colonies of Streptococcus faecalis and E. coli within 0.5 minutes at 0.5 PPM, utilizing section 9865.13 (Official Methods of Analysis [1990, 15th edition]) protocols of the AOAC. This is equivalent to the efficacy of chlorine at the same concentration.

More stringent testing was then performed in which the cultures were actually placed in trypticase soy nutrient broth, which allowed the pathogens being tested to replicate without being detached from its own food supply. Under these conditions, said oxide was able to achieve 100% kills on two strains of E. coli, namely, strains 10231 and 25254, at 2.5 PPM and Streptococcus faecalis strain 10541 at 5.0 PPM. Kills of 100% were also obtained with two other gram negative bacteria strains, Pseudomonas aeruginosa 9027 and Enterobacter cloacac 13047.

Silver tetroxide was further evaluated in analogous nutrient used for yeasts and molds, namely, Sabouraud dextrose broth. The vaginal yeast pathogen Candida albercans was totally killed at 2.5 PPM and that of the Saccharomycetpideae variety at 1.25 PPM.

Tests were conducted to see whether said tetroxide posed any harm to the human body. Accordingly, a 3% concentrate of the compound was prepared for a series of evaluations.

The first evaluation met the requirements of Code of Federal Regulations (40 CFR 160). It consisted of determining the single dose toxicity in rats or LD₅₀. All the animals survived so that the LD₅₀ was greater than 5000 mg./Kg. This was true for concentrations of compound of a magnitude of 6-60,000 times the actual concentrations that would be used in its utilization. This test classified the oxide as a category IV substance according to FDA protocols, or nontoxic.

The second evaluation was for acute dermal toxicity in rabbits. The protocol, 40 CFR 158.135, 81-2, was to determine the LD₅₀ for dermal application. All animals survived the maximum dose, 2000 mg./Kg., classifying the compound as category III with a dermal LD₅₀ greater than 2000 mg./Kg.

The third evaluation, entitled “Primary Dermal Irritation in Albino Rabbits”, conformed to 40 CFR 160. It consisted of exposing the rabbits for prolonged periods of time and observing edema, erythema, ulceration, necrosis and any other evidence of dermal reactions or tissue destruction. There were none, classifying the oxide concentrate as a category IV dermal agent by FDA criteria.

The fourth evaluation dealt with primary eye irritation. This also was in conformity with 40 CFR 160. There was absolutely no eye irritation when the crystal concentrate was applied, classifying it as a category IV substance with regard to eye effects according to FDA criteria.

Other objects and features of the present invention will become apparent to those skilled in the art when the present invention is considered in view of the accompanying examples. It should, of course, be recognized that the accompanying examples illustrate preferred embodiments of the present invention and are not intended as a means of defining the limits and scope of the present invention.

### DESCRIPTION OF THE PREFERRED EMBODIMENTS

As illustrative of the application and utilization of this invention in their preferred embodiments are the following:

### EXAMPLE I

Tetrasilver tetroxide (Ag₄O₄) crystals were prepared by modifying the procedure described by Hammer and Kleinberg in Inorganic Syntheses (IV, 12). A stock solution was prepared by dissolving 24.0 grams of potassium peroxydisulfate in distilled water and subsequently adding to this 24.0 grams of sodium hydroxide and then diluting the entire solution with said water to a final volume of 500 ml. Into 20 ml. vials were weighed aliquots of silver nitrate containing 1.0 g. of silver. Now 50 ml. of the aforementioned stock solution were heated in a 100 ml. beaker, and the contents of one of the vials was added to the solution upon attaining a temperature of 85° C. The beaker was then maintained at 90° C. for 15 minutes. The resulting deep black oxide obtained was washed and decanted four times with distilled water in order to remove impurities. The purified material was collected for further evaluation and comparison with commercial material. Commercial material was purchased from Johnson Matthey's Catalog, Chemicals Division, the Aesar Group, of Ward Hill, Massachusetts, under product code 11607 and generically listed in its Materials Safety Data Sheet as both silver peroxide and silver suboxide, having a purity of 99.9%.

Both the prepared and commercial materials were submitted for bactericidal evaluation following “good laboratory practice” regulations as set forth in Federal regulations (FIFRA and ffdca/40 CFR 150, May 2, 1984). The silver materials were tested as to whether they could kill pathogenic microorganisms with the intent of utilizing them in pharmaceutical applications. Once it could be determined that the devices inhibited a particular microorganism, the minimal concentration required of Ag₄O₄ was determined to inhibit the microorganism in nutrient broth. Strains of gram positive bacteria were chosen for the evaluation. One family of pathogens that are known for their deleterious effects on humans are popularly called “staph” infections. These infections are commonly contracted in hospitals having lax infectious screening procedures. Accordingly, three staph strains were selected as follows for evaluation: Staphylococcus aureus 9027 and 27543, and Staphylococcus epidermidis 12228. The inoculum nutrient broth was prepared according to AOAC specifications so as to contain 0.6-1 million organisms per drop of inoculum, each drop being equal to 0.05 ml. The broth itself was trypticase soy broth BBL 11766 prepared according to label instructions. Accordingly, the broth was prestandardized for the microorganisms in question in order to assure that the number of organisms remained constant within the margins of statistical allowance during the test period. Having carried out the procedures and having incubated the organisms for 24 hours at 34-35° C., it was found that staph organism 9027 was inhibited at 2.5 PPM, number 27543 at 5.0 PPM, and the 12228 organism at 0.625 PPM, all in the presence of 10 PPM sodium persulfate. This data was utilized to formulate a dermatological cream which would contain 100 PPM sodium persulfate and 10 PPM of oxide crystals to inhibit staph infections. The data was also utilized to formulate a surgical instrument sterilization formulation and a surgical scrub soap. Other gram positive bacteria pathogens were similarly evaluated. The minimal inhibiting concentration required for 100% kills are tabulated below for the organism in question as follows:

| | | | |
|---|---|---|---|
| Bacillus subtilis | 6633 | 5.00 | PPM |
| Micrococcus luteus | 9341 | 1.25 | PPM |
| Streptococcus ogalactiae | 27956 | 1.25 | PPM |
| Streptococcus pyogenes | 7958 | 2.50 | PPM |

The procedures described in Example I were analogously followed for the yeast pathogen Candida albicans using strain 16464 excepting that the nutrient broth was changed to Sabouraud dextrose broth (Difco 038217-9) to accommodate this yeast pathogen. It was found that 2.5 PPM of tetrasilver tetroxide completely inhibited the growth of this vaginal yeast infection. A gynecological cream was formulated against yeasts based on the results, as well as a cosmetic preservative.

As this invention may be embodied in several forms without departing from the spirit or essential characteristics thereof, the present embodiment is therefore illustrative and not restrictive, since the scope of the invention is defined by the appended claims rather than by the description preceding them, and all changes that fall within the metes and bounds of the claims or that form their functional as well as conjointly cooperative equivalents, are therefore intended to be embraced by these claims.

What is claimed is:

1. Pharmaceutical compositions comprising multivalent silver compounds in which the valence of silver in said compounds are 2 or 3, which compounds are capable of being employed as bactericides, viricides, algicides and fungicides.

2. Pharmaceutical compositions according to claim 1 in which the pathogenic efficacy of said composition is improved by the addition of an oxidizing agent.

3. Pharmaceutical compositions according to claim 1 where the multivalent silver compound is tetrasilver tetroxide (Ag₄O₄).

4. Pharmaceutical compositions according to claim 2 where the multivalent silver compound is tetrasilver tetroxide (Ag₄O₄).

5. Pharmaceutical compositions according to claim 2 where the oxidizing agent is persulfate.

6. A method of controlling the growth of bacteria, viruses, fungi and algae in pharmaceutical, cosmetic and chemical specialty products which comprises adding to said products a composition comprising multivalent silver compounds in which the valence of silver in said compounds are 2 or 3.

7. The method of claim 6 wherein the pathogenic efficacy of said composition is improved by the addition of an oxidizing agent.

8. The method of claim 6 wherein the multivalent silver compound is tetrasilver tetroxide (Ag₄O₄).

9. The method of claim 7 wherein the multivalent silver compound is tetrasilver tetroxide (Ag₄O₄).

10. The method of claim 7 wherein the oxidizing agent is a persulfate.

11. A pharmaceutical composition comprising a pharmaceutically effective amount of a multivalent silver compound in which the valence of silver in said compound is 2 or 3, which compound is capable of being employed as a bactericide, algicide and fungicide and an oxidizing agent for the multivalent silver compound which is present in an amount effective for activating the multivalent silver compound.

12. A pharmaceutical composition according to claim 11, where the multivalent silver compound is tetrasilver tetroxide (Ag₄O₄).

13. A method of controlling the growth of bacteria, fungi and algae in pharmaceutical products which comprises adding to said products a composition comprising a pharmaceutically effective amount of a multivalent silver compound in which the valence of silver in said compound is 2 or 3 and an oxidizing agent for the multivalent silver compound present in an amount effective for activating the multivalent silver the multivalent silver compound.

14. The method of claim 13, wherein the multivalent silver compound is tetrasilver tetroxide (Ag₄O₄).
